(19) United States
(12) Patent Application Publication
(10) Pub. No.: US 2008/0219930 A1
(43) Pub. Date: Sep. 11, 2008

(54) Method for the evaluation of dengue virus therapeutic agents

(76) Inventors: Timothy H. Burgess, Silver Spring, MD (US); Kevin R. Porter, Boyds, MD (US); Daniel A. Freilich, Washington, DC (US); Denise L. Doolan, Camp Hill (AU)

Correspondence Address: NAVAL MEDICAL RESEARCH CENTER;ATTN: (CODE 00L), 503 ROBERT GRANT AVENUE, SILVER SPRING, MD, 20910-7500, US

(21) Appl. No.: 11/507,322

(22) Filed: Aug. 21, 2006

Related U.S. Application Data
(60) Provisional application No. 60/709,804, filed on Aug. 22, 2005.

(51) Int. Cl. A61K 49/00

(52) U.S. Cl. 424/9.2

(57) ABSTRACT

The inventive subject matter relates to a method for evaluating potential compounds and vaccines for the prevention or treatment of dengue virus infection. The method utilizes pigs as an animal model for the evaluation of test vaccine or drug compounds. The breeds that can be utilized and in the inventive method include Yorkshire or Lancashire as well as miniature pig breeds.

### FIELD OF THE INVENTION

The inventive subject matter relates to a method for evaluating the immunogenicity and efficacy of vaccine or drug formulations against dengue virus using a pig or porcine cells as models of infection and pathogenicity.

### BACKGROUND OF THE INVENTION

Dengue fever, caused by a virus of the genus Flavivirus, is the most common human arbovirus infection worldwide and is of serious public health concern. Four antigenically distinct serotypes of dengue virus have been identified with all causing human diseases. Following infection, viremia is typically detected early at the onset of symptoms. Although most infections are mild, some infections result in dengue hemorrhagic fever and dengue shock syndrome. This usually occurs in a small number of people during a second infection with a dengue virus that is different from the virus causing the first infection (1).

Dengue and dengue hemorrhagic fever are found in most tropical areas including Africa, Asia, the Pacific, Australia and the Americas. Dengue virus infection occurs following the bite of dengue virus-infected Aedes mosquitoes that were previously infected by feeding on dengue-infected humans. Symptoms of dengue infection, including high fever, severe headache, retro-orbital pain, development of a rash, nausea, joint and muscle pain, usually start within five to six days following the bite of an infected mosquito. Symptoms of dengue hemorrhagic fever also include marked sub-dermal bleeding, causing a purplish bruise, as well as bleeding from the nose and gums. The fatality rate ranges from 1 to 30%, with most deaths occurring in infants.

Currently, the most effective prevention is through control of mosquito populations. No effective vaccines exist. A number of potential vaccine candidates currently exist, including subunit, whole virus and DNA vaccines. However, development of prophylactic methods and formulations have been hampered by the non-availability of suitable animal models. Studies using mice and non-human primates have yielded inconsistent results. Furthermore, in addition to the development of promising protective immunogens, the development of vaccine delivery strategies has been similarly hampered by the lack of adequate animal models. Additionally, immune enhancement of dengue infection, the mechanism of which is poorly understood due to a lack of a suitable animal model, is also a significant impediment to anti-dengue drug and vaccine development (2-6).

Vaccine studies on Japanese encephalitis virus (JEV), a member of the family Flaviviridae, an important animal pathogen, have been conducted using swine (7). These studies have demonstrated a reduction in JEV titers following immunization with attenuated vaccinia virus and plasmids engineered to express JEV genes. However, no similar animal models are known for dengue, also a member of the family Flaviviridae.

Because of the seriousness and widespread nature of dengue virus infection, effective prophylactic strategies are critically needed, especially vaccines. Vital to the development of these strategies is the development of suitable animal models.

### SUMMARY OF THE INVENTION

To address the lack of a suitable animal model and methods for the development and evaluation of dengue virus prophylactic measures, it has been discovered that pigs (sus scrofa), including Yorkshire and Lancashire breeds, as well as miniature pigs, including the Yucatan (Mexican hairless) minipig, can develop viremia in response to dengue virus infection as well as mount an immune response to dengue antigens. Therefore, due to the suitability of this species to serve as animal models for dengue studies, an aspect of this invention is the use of this species in methods evaluating anti-dengue virus therapeutic approaches. Minipigs and micropigs are especially well suited due to the lower cost in husbandry. The inventive animal model is useful for evaluating the activity of compounds of potential use in treating active dengue infection and for evaluating anti-dengue vaccines.

Therefore, an aspect of the invention is a method for determining the activity of a compound that includes administering the test compound prior to or after infection of pigs with dengue virus. Analysis of the test compounds effectiveness is then made by evaluating the ensuing disease pathology and symptomatology of the dengue-infected pigs as well as immune response to the test compound, in the case of vaccine candidates.

### BRIEF DESCRIPTION OF THE DRAWINGS

FIG. 1. IgM response after inoculation with dengue-1 virus.

FIG. 2. IgG response after inoculation with dengue-1 virus

FIG. 3. Photograph of a hemorrhagic rash representing a disease manifestation of dengue virus infection

FIG. 4. Photomicrograph of a skin biopsy of a hemorrhagic rash occurring after a second dengue virus infection

FIG. 5. ELISA results following immunization of minipigs with dengue-1 DNA vaccine

FIG. 6. Neutralizing antibody titer following administration of dengue-1 DNA vaccine

FIG. 7. Elispot data showing T cell response following administration of dengue-1 DNA vaccine

### DETAILED DESCRIPTION OF THE PREFERRED EMBODIMENTS

The present invention centers on a critical need for a dengue virus animal model useful for the design and evaluation of compounds for the prevention and treatment of dengue infection. The current invention addresses this need by the discovery that the pigs, such as the Yorkshire and Lancashire breeds, as well as miniature pigs, can develop viremia in response to dengue virus infection as well as mount an immune response to dengue antigens.

Although Yorkshire or Lancashire breeds of pigs are readily available and inexpensive, miniature swine (minipigs) were developed over the past fifty years to provide conveniently-sized pigs for experimentation. Several breeds of minipigs exist. The Yucatan (Mexican hairless) pig is the only naturally occurring minipig and is native to Central America. This breed served as the foundation for several other breeds of minipigs, including the Hormel pig, the Panepinto micropig, the Goettingen miniature pig and the Vietnamese pot-bellied pig.

Pigs, including miniature swine such as Yucatan minipigs, possess other important characteristics that make this species particularly amenable to dengue virus studies. The list of characteristics include physiological similarity to humans; similar human doses are applicable to pigs; delivery systems applicable to humans can be used with the pig model; large numbers of peripheral blood mononuclear cells can be harvested; lower cost compared to non-human primate models, especially in the use of minipigs.

An embodiment of the invention is the testing of compounds aimed at either treating dengue infection and disease or evaluating the efficacy of anti-dengue vaccine components. The method includes:
a) administering a test compound to a pig such as a Yorkshire or Lancashire breed or a miniature pig, such as the Yucatan minipig, before or after infection with dengue viral strains;
b) evaluating ensuing symptomotology of infected pigs compared to control animals not receiving the compound or receiving control compounds or minipigs that have not been infected;
c) evaluating disease state by histological methods;
d) evaluating dengue virus titer by immunological methods, such as enzyme-linked immunosorbent assay (ELISA) or by molecular methods such as polymerase chain reaction (PCR);
e) evaluating the ability of a compound to reduce the level of viremia after a challenge with live dengue virus.

Another embodiment of the invention is the testing and evaluation of potential vaccines against dengue virus. The method includes:
a) administering one or more doses of a test compound to a pig such as a Yorkshire or Lancashire breed or a miniature pig, such as the Yucatan minipig, before or after infection with dengue viral strains;
b) obtaining peripheral blood mononuclear cells (PBMC) and serum to assess viremia after live virus injection;
c) evaluating surface expression of immune markers and cytokine gene expression by the peripheral blood mononuclear cells;
d) evaluating dengue disease of vaccinated, infected pigs compared to control animals that have either not been infected or that have not received the vaccine;
e) evaluating the ability of a vaccine to reduce the level of viremia after a challenge with live dengue virus.

The following examples are presented to permit a better understanding of the features and advantages of the invention. The examples, however, are not to be construed as limiting the invention.

### Example 1

### Induction of Viremia in Pigs

Optimal testing of anti-dengue virus activity by test compounds requires the ability to induce viremia in an animal model. Viremia in pigs was demonstrated by subcutaneous inoculation of dengue-1 (Western Pacific 74, WP74) virus to pigs. Two groups of pigs received either a low dose of 10⁵ plaque-forming units of dengue-1 virus (group 1) or 10⁷ plaque-forming units of dengue-1 virus (group 2) (Table 1). A third group received no virus (group 3). Serum from whole blood was obtained prior to virus inoculation and daily thereafter for 14 days in order to assess viremia by tissue culture isolation in Vero and C6/36 cells. Blood is easily obtained from pigs, including miniature pigs via the cranial vena cava and this site is amenable to daily needlesticks. As shown in Table 1, all animals administered virus developed at least one day of viremia detectable by tissue culture isolation. Virus was not detected from sera of control animals (not shown). Confirmatory evidence of virus was also obtained by reverse-transcriptase polymerase chain reaction (RT-PCR). Overall, there was an 83% concordance between RT-PCR and virus isolation. Quantitative RT-PCR suggested that there was a low concentration of virus in blood. Although it is possible that virus titers were low in these animals, the low virus titers observed by RT-PCR may be explained by previous observations of nonspecific inhibition of reverse transcriptase by pig serum.

TABLE 1
Dengue-1 viremia induction in pigs

| | Low-dose of dengue-1 | | | | High-dose of dengue-1 | | | |
|---|---|---|---|---|---|---|---|---|
| | virus adiminstered (group 1) | | | | virus administered (group 2) | | | |
| Day | Pig 1 | Pig 2 | Pig 3 | Pig 4 | Pig 5 | Pig 6 | Pig 7 | Pig 8 |
| 0 | − | − | − | − | − | − | − | − |
| 1 | + | − | − | + | + | + | + | − |
| 2 | − | − | − | − | + | − | − | + |
| 3 | + | − | − | + | + | + | + | + |
| 4 | + | + | + | + | − | + | + | + |
| 5 | + | − | − | + | + | + | + | − |
| 6 | − | − | − | − | + | + | + | − |
| 7 | + | − | − | + | − | + | + | − |
| 8 | + | − | − | + | − | + | + | − |
| 9 | − | − | − | + | + | + | + | − |
| 10 | − | − | − | − | − | + | + | + |

By virus isolation, the mean number of days of viremia were 3.75 and 6.75 after the first inoculation in the low and high groups, respectively.

When these animals were inoculated a second time, six months after the first inoculation, there was an 80% reduction in the mean number of days of viremia compared to the control animals the were inoculated for the first time. Following this second inoculation, the mean number of days of viremia was 1 for both the low and high dose groups (data not shown). For control animals that received their first virus inoculation, the mean number of days of viremia was 5.

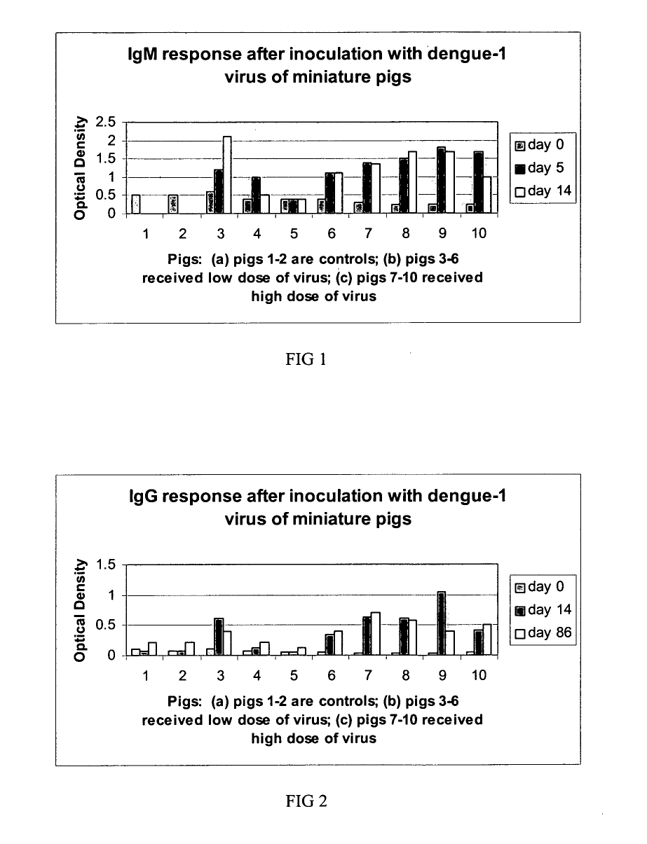

Antibody response to dengue-1 infection was measured by enzyme-linked immunosorbent assay (ELISA). Both IgM (FIG. 1) and IgG (FIG. 2) responses in dengue-1 inoculated pigs were clearly demonstrable in a dose-response fashion. For both IgM and IgG, dengue inoculated pigs developed responses in 2 of 4 low-dose animals and 4 of 4 high dose animals. Maximum IgM antibody responses were typically observed on day 5 after inoculation in animals receiving a high-dose of dengue-1 inoculum (10⁷ plaque-forming units) (FIG. 1). In pigs receiving low-doses of dengue-1 inoculum (10⁵ plaque-forming units) maximum IgM antibody responses were observed either at day 5 or day 14 after inoculation. In both high and low-dose animals, IgM antibody was detectable at least as early as 5 days after inoculation. Similarly, IgG antibody was observed at maximal or near maximal levels as early as 14 days after inoculation (FIG. 2).

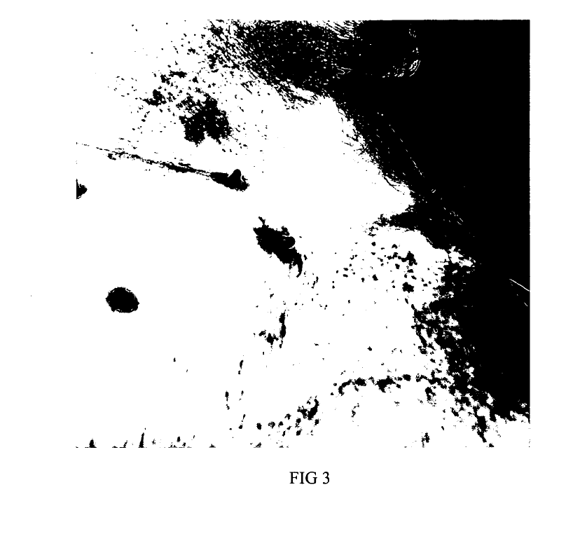

In addition to antibody responses, the majority of inoculated pigs receiving a second experimental inoculation with dengue virus developed an erythematous, maculopapular skin eruption that was petechial in appearance at approximately day 4 post inoculation (FIG. 3). No rash was seen after only a single inoculation. The rash was widely distributed, involving the axilla, groin and post-auricular regions and in most animals the back and in some animals diffusely involving the abdomen. The rash resolved within a few days to a week.

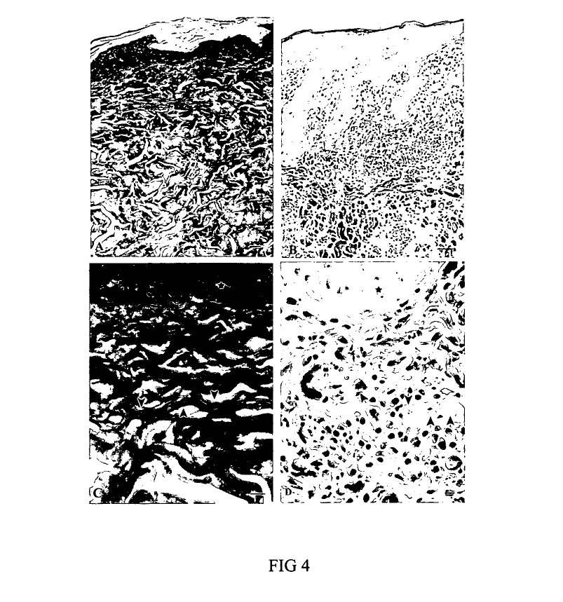

FIG. 4 shows the skin biopsy results taken from the site of the skin rash of one animal compared to that of normal pig skin. Panel A represents normal skin. The section is oriented with the epidermis at the top. The dermis typically contains abundant dense collagenous connective tissue. Panel B is a skin sample from the rash. There is marked expansion of the dermis and separation of the dermal collagen bundles by edema fluid and infiltrates of mononuclear cells, neutrophils and eosinophils. The overlying epidermis is hyperplastic and edematous. Panel C represents normal skin at a higher magnification. This higher magnification demonstrates the normally inconspicuous vessels of the superficial dermis (arrowheads) and dense fibrous connective tissue, a normal feature of pig skin epidermis (*). Panel D is a higher magnification of the skin rash. There is endothelial hypertrophy and thickening of the vessel walls within the superficial dermis (arrowheads). The perivascular spaces are expanded by clear edema fluid and infiltrates of inflammatory cells. The basal cell layer of the epidermis (*) is hyperplastic. Taken together, the histological findings of the skin rash are similar to the histological findings that occur with dengue hemorrhagic fever in humans.

### Example 2

### Evaluation of Induced Immune Cell Response Following Administration of Test DNA Vaccine

An example of the utility of the present invention is the evaluation of promising vaccines. DNA vaccines are particularly important as potential vaccine candidates for dengue virus. In this example, a plasmid was constructed encoding dengue-1 proteins. In these studies, the DNA vaccine was administered to pigs in multiple doses in different dose sizes. Control animals are also included that received saline diluent without plasmid. Different routes of immunization are also employed in different animals including intradermal (ID) and intramuscular (IM) injection. Although the current example utilized miniature pigs, other breeds of pigs, including larger breeds, are contemplated as also being suitable for use.

In this example, 30 animals were used, including four groups of six animals receiving vaccines. Groups 1 and 2 received 1 mg of DNA vaccine intramuscularly (IM) and intradermally (ID), respectively. Groups 3 and 4 received 5 mg of DNA vaccine IM and ID, respectively. A fifth group was included as a control. Immunogenicity determinations were made at 2 weeks after and 30 weeks after the third dose of a 3-dose vaccination regimen via IM and ID administration routes. At various time points following administration of the DNA vaccine, serum and PBMC were removed from the minipigs for evaluation of the specific induction of B and T cell immune responses.

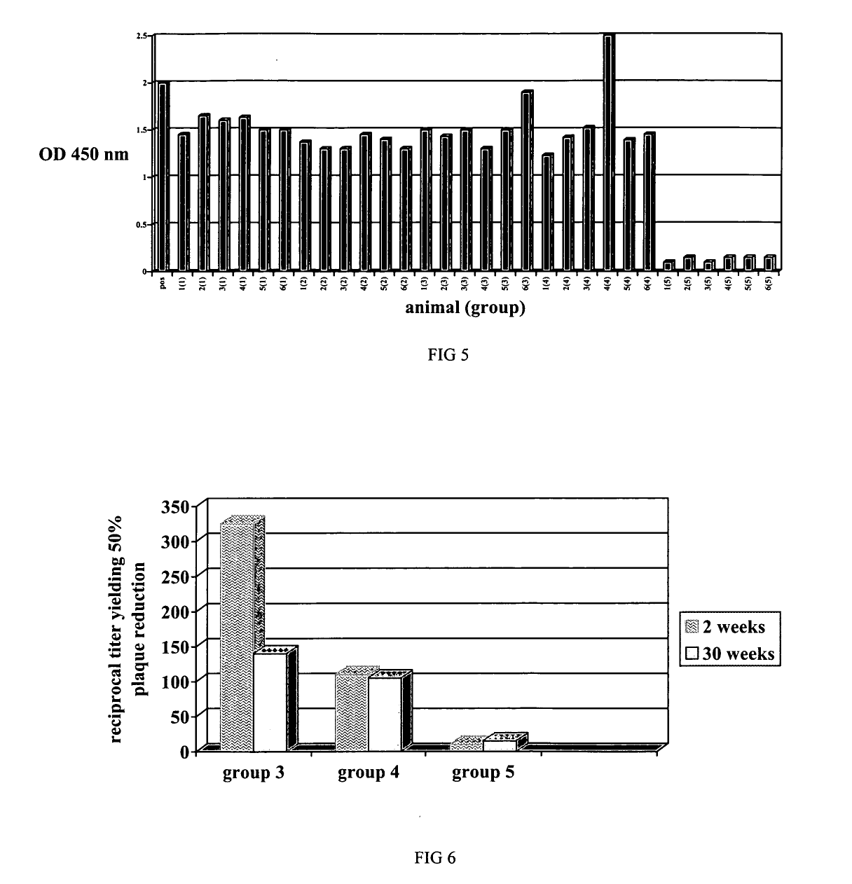

Antibody responses were measured by ELISA and by in vitro neutralization assays in the minipigs. The minipigs evaluated demonstrated excellent immune responses to the DNA vaccine. As shown in the ELISA results of FIG. 5, antibody was induced in all minipigs and remained detectable after 7 months in the animals receiving 5 mg per dose. In FIG. 5, the number in parenthesis refers to the group to which the individual minipig was a member. Furthermore, antibody neutralization study results, shown in FIG. 6, show that all groups produced dengue neutralizing antibody.

Analysis of cytokine gene expression is an important indicator of specific T cell activation and thus a valuable indicator of host immunity to a specific antigen or vaccine. In vitro analysis of T cell activity is therefore a useful predictive factor regarding the potential efficacy of the test compound as a vaccine. Although a number of techniques are available to accurately assess cytokine gene activation, ELISPOT and intracellular cytokine staining are particularly useful.

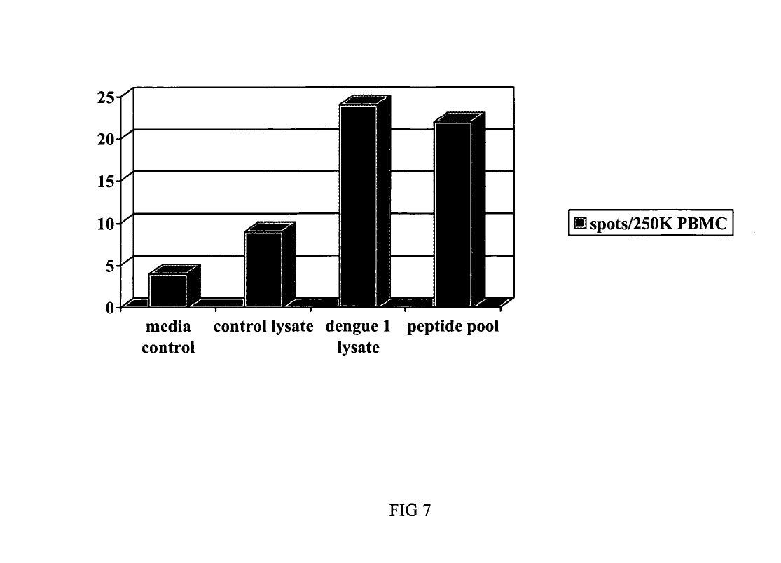

The determination of in vitro T cell responses to dengue virus was determined using dengue virus lysates or pools of peptides encompassing the dengue antigen encoded by the plasmid as antigen. In this example, ELISPOT analysis on swine PBMC's was carried out using either frozen or fresh PBMCs collected from minipigs. In either case, porcine PBMCs are plated in anti-cytokine-antibody-coated microtiter plates at varying cell densities and stimulated with mitogens (e.g. conconavalin A) or specific antigen. Cells secreting IFNγ were enumerated by detection with chromogen-labeled anti-cytokine antibodies. Optimal results are obtained using mouse anti-porcine IFNγ antibody at 10 μL and a PBMC density of 250,000 per well in MAIP™ plates (Millipore, Billerica, Mass.) with 3 amino-9-ethyl carbozole (AEC) chromogen. Minipigs receiving the DNA vaccine IM displayed significant post-vaccination T cell activity in response to dengue antigen, as evidenced by ELISPOT assay (FIG. 7).

Intracellular cytokine staining can be accomplished on PMBC's or purified immune cells collected from minipigs before or at various time points subsequent to administration of test compounds. At various time points, collected immune cells expressing CD3+, CD4+ and CD8+T cell subsets and co-expressing intracellular levels of important cytokines such as IFNγ, TNFα and IL-8 are enumerated after stimulation with specific antigen, in vitro. The expression is compared to controls including expression of the markers following stimulation, in vitro with lectins and expression of cells that have not been activated in vitro.

### REFERENCES

1. Halstead, S. B. 1997. Epidemiology of dengue and dengue hemorrhagic fever. In Dengue and Dengue Hemorrhagic Fever. D. J. Gubler and G. Kuno, eds. Cab International, London. 23-44.
2. Burke, D. S., A. Nisalak, D. E. Johnson, and R. M. Scott. 1988. A prospective study of dengue infections in Bangkok. Am. J. Trop. Med. Hyg. 38: 172.
3. Kliks, S. C., A. Nisalak, W. E. Brandt, L. Wahl, and D. S. Burke. 1989. Antibody-dependent enhancement of dengue virus growth in human monocytes as a risk factor for dengue hemorrhagic fever. Am. J. Trop. Med. Hyg. 40:444.
4. Halstead, S. B., and E. J. O'Rourke. 1977. Antibody-enhanced dengue virus infection in primate leukocytes. Nature. 265:739.
5. Halstead, S. B., E. J. O'Rourke, and A. C. Allison. 1977. Dengue viruses and mononuclear phagocytes. II. Identity of blood and tissue leukocytes supporting in vitro infection. J. Exp. Med. 146::218.
6. Konishi, E., S. Pincus, E. Paoletti, W. W. Laegreid, R. E. Shope, and P. W. Mason. 1992. A highly attenuated host range-restricted vaccinia virus strain, NYVAC, encoding the prM, E, and NS1 genes of Japanese Encephalitis virus prevents JEB viremia in swine. Virology 190: 454.

What is claimed is:

1. A method for identifying a compound with potential for use in the treatment of dengue virus infection comprising:
a. administering a test compound to a pig infected with dengue virus; and
b. determining whether the test compound inhibits dengue virus infection in said pig.

2. The method for identifying a compound with potential for use in the treatment of dengue virus infection as in claim 1 further comprising:
c. administering a control compound to a pig infected with dengue virus; and
d. determining whether said test compound inhibits dengue virus more than said control compound.

3. The method for identifying a compound with potential for use in the treatment of dengue virus infection as in claim 1 wherein said pig is selected from the group consisting of Yorkshire breed, Lancashire breed, Yucatan minipig, Panepinto micropig and Goettingen miniature pig.

4. The method for identifying a compound with potential for use in the treatment of dengue virus infection as in claim 1 wherein said compound is an anti-viral drug.

5. A method of identifying a compound with potential for use as an anti-dengue vaccine comprising:
a. administering a test compound to a dengue virus infected pig; and
b. administering a test compound to a pig and then infecting the pig with dengue virus;
c. determining whether the test compound induces anti-dengue virus infection immune response in said pigs.

6. The method of identifying a compound with potential for use as an anti-dengue vaccine as in claim 5 further comprising:
d. administering a control compound to dengue virus infected pig;
e. administering a control compound to a pig and then infecting the pig with dengue virus; and
f. determining whether said test compound produces a larger anti-dengue immune response than said control compound in said pigs.

7. The method of identifying a compound with potential for use as an anti-dengue vaccine as in claim 5 wherein said pig is selected from the group consisting of Yorkshire, breed, Lancashire breed, Yucatan minipig, Panepinto micropig and Goettingen miniature pig.

8. The method of identifying a compound with potential for use as an anti-dengue vaccine as in claim 5 wherein said test vaccine compound is a DNA vaccine.

9. The method of identifying a compound with potential for use as an anti-dengue vaccine as in claim 5 wherein said test compound is a polypeptide.
